(19) United States
(12) Patent Application Publication
(10) Pub. No.: US 2009/0292295 A1
(43) Pub. Date: Nov. 26, 2009

(54) Umbilical cord ligature device

(76) Inventor: Armando Garcia-Alonso Peñuñuri, Sonora (MX)

Correspondence Address: Mark Ogram, 6990 E. 22nd Street, Ste. 110-186, Tucson, AZ, 85710, US

(21) Appl. No.: 11/597,205

(22) PCT Filed: Jul. 5, 2005
(86) PCT No.: PCT/MX2005/000050

§ 371 Date: Jan. 23, 2009

(51) Int. Cl. A61B 17/42

(52) U.S. Cl. 606/120

(57) ABSTRACT

An umbilical cord ligature device comprising a flexible latex ligature that is pre-assembled on a semi-rigid plastic ring. The umbilical cord is slipped through the device to a point close to the baby's abdominal wall, and with a twist of fingers, the latex ligature is then released from the ring over the umbilical cord, where it stays compressing the cord until it dehydrates and falls off. The pressure that the ligature exerts around the umbilical cord during its dehydration period makes hemorrhages virtually impossible.

### OBJECT OF THE INVENTION

The invention relates to an umbilical cord ligature device. The object of the invention is to ligate the umbilical cord and assure hemostasis in a more simple, effective and safe way. Also, it is an object of the invention to provide an improved umbilical cord ligature that is aesthetically more pleasing, small and discrete.

### BACKGROUND AND FIELD OF THE INVENTION

At birth, the separation of the baby from the mother's placenta is done by applying two surgical clamps to the umbilical cord and then severing the cord between the clamps. Subsequently, the pediatrician, physician or nurse who takes care of the baby, ligates the umbilical cord in a point close to the baby's abdominal wall using a Plastic Clamp or an Umbilical Tape. Once the umbilical cord is ligated, the excess cord is cut off and discarded.

Those are the two traditional methods for ligating the umbilical cord of infants and some mammal newborns that, as humans, require an umbilical cord ligation after birth; even though, these methods have disadvantages: the Umbilical Tape is a non elastic cotton string that is used to tie the umbilical cord with a square knot which exerts static pressure onto the cord. This static pressure ceases as the umbilical cord shrinks during its dehydration period, permitting hemorrhages to occur. In addition, it is a disadvantage of the Umbilical Tape that it stains and gets dirty with baby's excretions, so it needs to be occasionally replaced.

The other method is to use a Plastic Umbilical Clamp, which is very effective and produces a safe and reliable ligation of the umbilical cord. However, because of its large size and bulky shape, the Plastic Umbilical Clamp is likely to cause accidental tractions to the umbilical cord, as a result it reduces the baby's comfort. In addition, this device is cumbersome for the mother to handle and clean the baby, and is aesthetically unattractive.

A number of clamps are known in the art for clamping the umbilical cords of newborn babies. Perhaps the most common clamp currently used for such purposes is the umbilical cord clamp disclosed in U.S. Pat. No. 4,212,303, issued to John L. Nolan on Jul. 15, 1980 and assigned to “Hollister Incorporated”.

Other devices have been developed to clamp and sever the umbilical cord in one motion, like one disclosed in U.S. Pat. No. 5,584,840 to Ramsey et al on Dec. 17, 1996; and also solicitude No. 9409371 to Alfonso Ortega Medina, presented to the Mexican Institute of Industrial Property. The Umbilical Cord Clamp and Cutter device is not commonly used; it is more expensive than the Plastic Clamps and usually has a complex design that makes it difficult to use.

### DETAILED DESCRIPTION OF THE INVENTION

The invention consists of a small and novel umbilical cord ligature device that permits to effectively close all blood vessels in the umbilical cord and makes hemorrhages virtually impossible. The device gives confidence to the pediatrician because once applied, the device assures hemostasis of the umbilical cord, and it doesn't need to be replaced when the cord shrinks during its dehydration period.

Once applied, the ligature of the device looks very natural and discrete, and it fully permits the baby's cleaning procedure since it is small, soft, flexible and can be washed with water and soap without affecting its elastic properties. It increases the baby's comfort and its mother's confidence because the device eliminates the risk of accidental tractions to the umbilical cord due to its small size.

More specifications of the invention are fully explained in further pages of this document. The description has two parts: first comes a detailed description of the preferred embodiments of the device, and the second part describes how the invention is used.

### DETAILED DESCRIPTION OF THE PREFERRED EMBODIMENT

In the preferred embodiment, the device is formed by two parts:
1. The ligature: A cylindrical ligature made of latex, or any other flexible and retractile material. Ideally, it has an external diameter of 6.35 millimeters, a wall thickness of 1.3 millimeters and a height of 5.0 millimeters.
2. The ring: A semi-rigid but flexible plastic ring with a preferred external diameter of 1.9 centimeters, a wall thickness of 2.9 millimeters and a height of 6.0 millimeters. It also has an opening slit in its wall permitting the ring to flex and twist.

Through a mechanical force, the ligature is expanded and assembled on the exterior surface of the ring; therefore both pieces form the device. Once assembled, the device is ready to make a one-way movement ligation of the umbilical cord in the right place.

Before it's use, the device is submitted to Ethylene Oxide sterilization, or any other sterilization procedure that doesn't affect the physical properties of the different fabrication materials.

### How the Invention is Used

Once sterile, the device is ready to be used by the pediatrician, physician or any person who is in charge of the baby. The physician must pass the umbilical cord through the ring of the device and locate it at the desired distance from the baby's abdominal wall (usually 1 to 1 ½ inches), and with a twist of fingers release the ligature from the ring over the umbilical cord and discard the ring. Once applied the ligature tightens around the cord and the physician then cuts and discards the remaining distal part of the cord.

The ligature will continue tightening the cord for the rest of its dehydration period (7 to 10 days), making hemorrhages virtually impossible. The pressure that the ligature exerts over the umbilical cord maintains the blood vessels sealed all the time and prevents any leakage of blood out of them.

### Other Uses of the Invention

This invention, which characteristics have been previously described in detail, has a primary field of application in medicine. Another field of application of this invention would be veterinarian medicine, because there are several mammal newborns that, as humans, require an umbilical cord ligation after birth.

Due to its simple design, this invention can be used by any physician, pediatrician or nurse or veterinarian, since its application doesn't require advanced surgical skills from the user.

In addition, the device of the present invention has an industrial application because it can be manufactured at a cost low, lower than the existent clamps and different methods for closing the umbilical cord.

### BRIEF DESCRIPTION OF THE DRAWINGS

FIG. 1 is a perspective view of the ligature before it is expanded and assembled on the ring.

FIG. 2 is a perspective view of the ring, showing its wall opening slit that should be previously colored to indicate its location.

FIG. 3 is a perspective view of the ring of FIG. 2 in an open position showing its flexibility.

FIG. 4 is a perspective view of both, ligature and ring, already assembled and forming the complete device.

FIG. 5 is a lateral view of the apparatus shown in FIG. 4.

FIGS. 6, 7 and 8 show how the umbilical cord should be passed through the device and how the ligature will be liberated on it.

FIG. 9 shows how the ligature stays on the umbilical cord while the ring is discarded.

FIG. 10 shows the umbilical cord after the procedure.

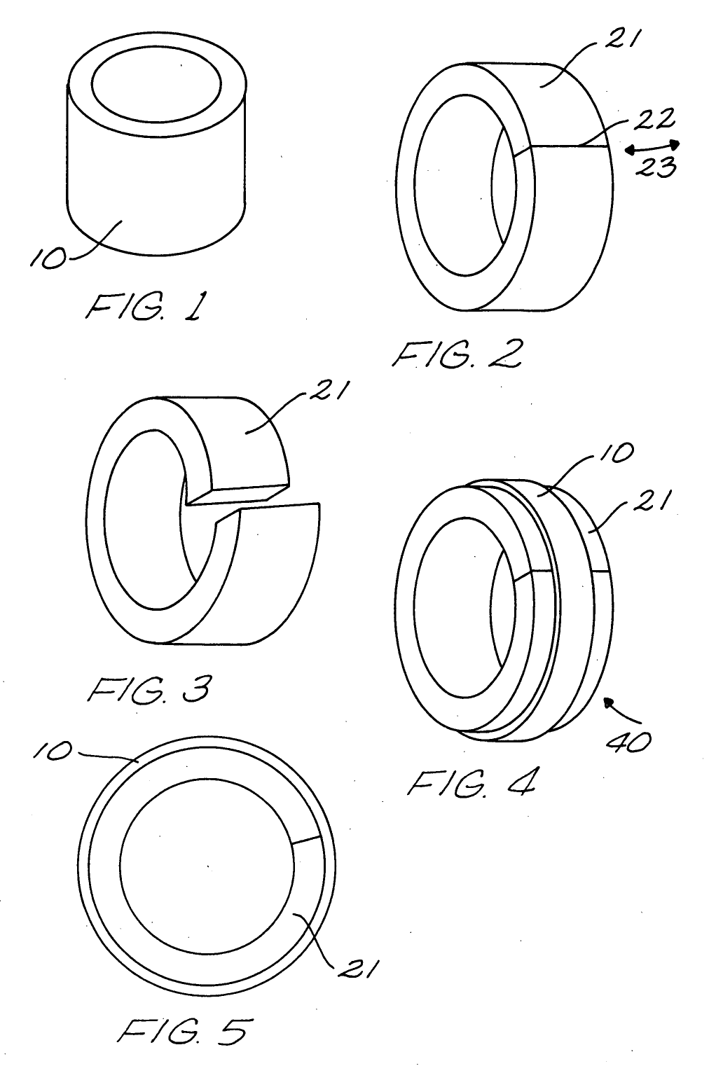

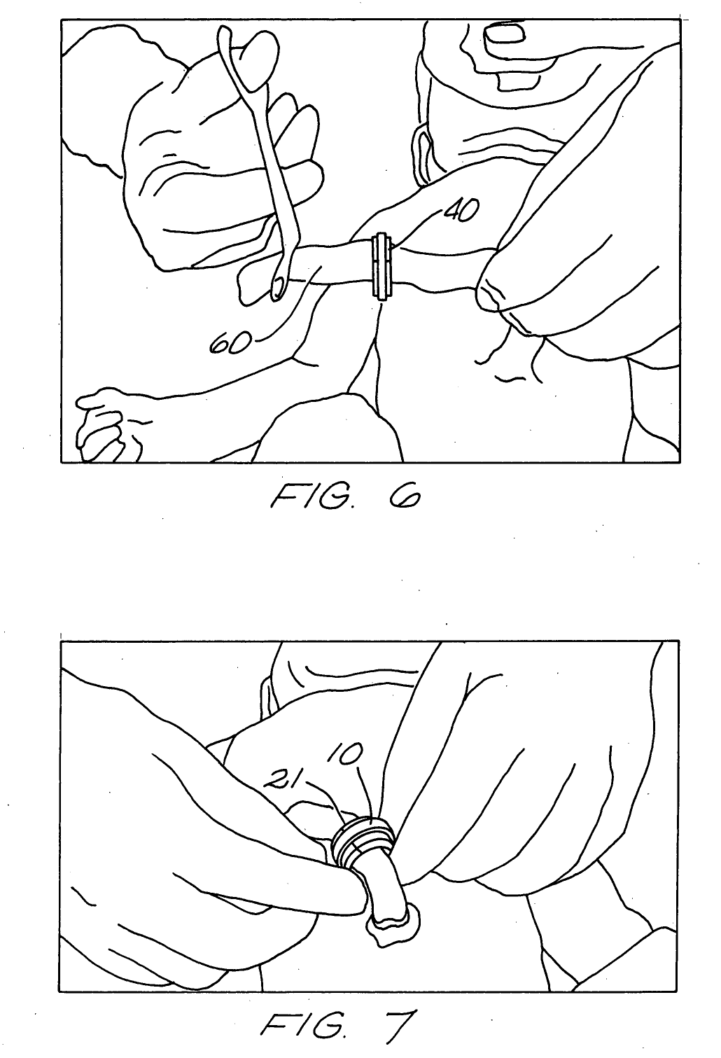

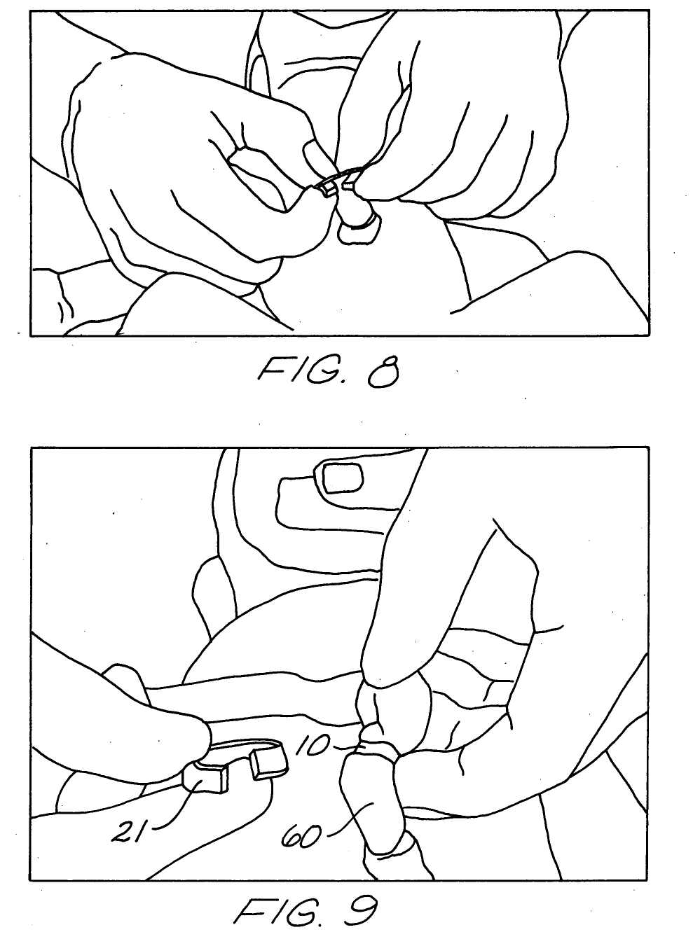

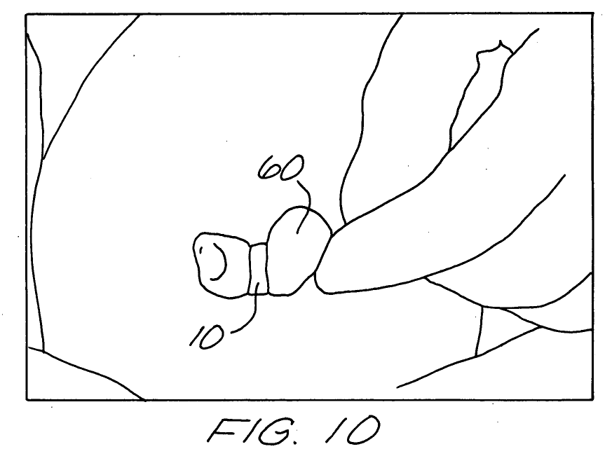

What is claimed is:

1. A device for ligating an umbilical cord, said device comprising: a ligature assembled on the external surface of a ring; wherein, by liberating said ligature in one continuous motion while said ring is positioned on an umbilical cord, said ligature compresses and closes the umbilical cord thereby assuring hemostasis and preventing fluid leakage.

2. The device of claim 1, wherein said ligature and said ring comprise a single integral structure.

3. The device of claim 1, wherein said ligature is formed of a retractile material.

4. The device of claim 1, wherein said ring is a semi-rigid material.

5. The device of claim 1, wherein said ring has an opening slit therein.

6. An assembly for ligating an umbilical cord comprising:
a) a ring having an internal opening larger than an umbilical cord; and,
b) a ligature assembled on an external surface of said ring.

7. The assembly according to claim 6, wherein said ring has a slit therethrough.

8. The assembly according to claim 7, wherein, said ring is configured to liberate said ligature from the external surface of said ring in one continuous motion such that said ligature closes the umbilical cord to assure hemostatis and prevent fluid leakage or flow therefrom.

9. The assembly according to claim 8, wherein said ligature is formed of a retractile material.

10. The assembly according to claim 9, wherein said retractile material includes latex.

11. An assembly for ligating an umbilical cord comprising:
a) a ring having an internal opening larger than an umbilical cord, said ring having a slit therethrough such manual twisting pressure thereon distorts said ring at said slit; and,
b) a ligature stretched around an external surface of said ring.

12. The assembly according to claim 11, wherein, said ring is configured to liberate said ligature from the external surface of said ring when manual twisting pressure is applied to said ring.

13. The assembly according to claim 12, wherein said ligature and said ring are sterilized as a unit.

14. The assembly according to claim 13, wherein said ligature is formed of a retractile material.

15. The assembly according to claim 14, wherein said retractile material includes latex.

16. The assembly according to claim 13, wherein said ring is formed of a semi-rigid material.

17. The assembly according to claim 16, wherein said semi-rigid material includes polycarbonate.

18. An assembly for ligating an umbilical cord comprising:
a) a slit ring;
b) an elastic ligature stretched onto an external surface of said ring; and, wherein said ligature and said ring are sterilized in an assembled state.

19. The assembly according to claim 18, wherein bending said slit ring at said slit liberates said ligature from the external surface of said slit ring.
